(19) United States
(12) Patent Application Publication
(10) Pub. No.: US 2014/0371589 A1
(43) Pub. Date: Dec. 18, 2014

(54) SUBJECT INFORMATION OBTAINING APPARATUS

(71) Applicant: CANON KABUSHIKI KAISHA, Tokyo (JP)

(72) Inventor: Takaaki Nakabayashi, Kawasaki-shi (JP)

(21) Appl. No.: 14/301,194

(22) Filed: Jun. 10, 2014

(30) Foreign Application Priority Data
Jun. 13, 2013 (JP) 2013-125065

(51) Int. Cl. A61B 8/00; A61B 8/08

(52) U.S. Cl. CPC A61B 8/406; A61B 8/0825
USPC 600/442; 600/459

(57) ABSTRACT

A subject information obtaining apparatus includes a shape detector configured to detect the shape of a holding member holding a subject and a shape controlling unit configured to control the shape of the holding member based on result of a detection by the shape detector.

### BACKGROUND

1. Field

Aspects of the present invention generally relate to subject information obtaining apparatuses that receive elastic waves which travel through subjects.

2. Description of the Related Art

Some known subject information obtaining apparatuses that receive elastic waves, such as acoustic waves and ultrasonic waves, include an ultrasonic apparatus that transmits and receives an ultrasonic wave with a probe and turns the ultrasonic wave into an image, and an apparatus that, utilizing photoacoustic effects, receives, with a probe, an acoustic wave (typically, an ultrasonic wave) generated as a subject is irradiated with light and turns the acoustic wave into an image.

There apparatuses employ a technique in which a subject (breast) is measured while the subject is being pressurized to have its thickness reduced. For example, Japanese Patent Laid-Open No. 2007-282960 describes an ultrasonic diagnostic apparatus in which a sealed structural body formed by a film and a container is filled with an acoustic matching liquid and a breast is measured while being held, in a pressurized manner, by the film portion of the sealed container. Japanese Patent Laid-Open No. 2007-282960 indicates that the stated apparatus can apply a predetermined pressure (fixed pressure) regardless of the size of the breast by modifying the amount of the matching liquid or by modifying the tensile force of the thin film and can shape the breast so as to be suitable for measurement. Specifically, in a case in which the size of the breast is small, the amount of the matching liquid is increased or the tensile force of the film is increased so as to increase the pressure on the breast. Meanwhile, in a case in which the size of the breast is large, the amount of the matching liquid is decreased or the tensile force of the film is reduced so as to reduce the pressure on the breast. Through such an adjustment, the predetermined pressure (fixed pressure) is applied to the breast regardless of the size of the breast. Japanese Patent Laid-Open No. 2007-282960 also indicates that the predetermined pressure can be applied on the breast more precisely by measuring the tensile force of the film.

The apparatus disclosed in Japanese Patent Laid-Open No. 2007-282960, however, has shortcomings in terms of a signal-to-noise (S/N) ratio of a signal received from the subject (breast), and an improvement has been expected. To be more specific, with the apparatus described in Japanese Patent Laid-Open No. 2007-282960, there may be a case in which the thickness of the subject is not reduced even when the measured tensile force is sufficient, if the size of the breast is large or if the breast is firm. Thus, the stated apparatus has an issue in that, in a case in which a site on which information is to be obtained is located deep inside the subject, the S/N ratio may decrease due to the attenuation of the ultrasonic wave.

### SUMMARY

An aspect of the present invention is generally directed to suppressing attenuation of an acoustic wave through a holding member and a subject so as to improve an S/N ratio by detecting and controlling the shape of the holding member holding the subject.

According to an aspect of the present invention, a subject information obtaining apparatus includes a holder configured to hold a subject, a receiver configured to receive, through the holder, an elastic wave emitted from the subject held by the holder, at least one shape detector configured to detect a shape of the holder, and a shape controlling unit configured to control the shape of the holder based on a result of detection by the at least one shape detector.

Further features of the present disclosure will become apparent from the following description of exemplary embodiments with reference to the attached drawings.

### BRIEF DESCRIPTION OF THE DRAWINGS

FIG. 1 schematically illustrates a configuration of a subject information obtaining apparatus according to a first exemplary embodiment.

FIGS. 2A and 2B schematically illustrate a configuration in which a plurality of shape detectors is provided.

FIG. 3 illustrates a configuration of a probe unit.

FIGS. 4A and 4B illustrate states of a holding member, respectively, before and after detecting and controlling the shape of the holding member.

FIG. 5 schematically illustrates a configuration in which a plurality of shape detectors is provided.

FIGS. 6A and 6B schematically illustrate a configuration that allows the position at which the shape of the holding member is detected to be changed.

FIGS. 7A and 7B schematically illustrate a configuration of a subject information obtaining apparatus according to a second exemplary embodiment.

FIGS. 8A and 8B schematically illustrate a configuration of a subject information obtaining apparatus according to a third exemplary embodiment.

FIG. 9 schematically illustrates a configuration for measuring a distance with an ultrasonic probe.

### DESCRIPTION OF THE EMBODIMENTS

Hereinafter, exemplary embodiments will be described with reference to the drawings. It is to be noted that the sizes, the materials, the shapes, and so forth of components described hereinafter should be modified, as appropriate, in accordance with the configuration of an apparatus to which the exemplary embodiments are applied or with various other conditions, and these exemplary embodiments are not seen to be limiting.

Hereinafter, an exemplary apparatus that obtains information on a subject by irradiating the subject with light and by receiving, with a probe, an acoustic wave (typically, an ultrasonic wave) generated in the subject will be described in each of the exemplary embodiments. The information to be obtained on the subject includes characteristic information that reflects an initial sound pressure distribution of an acoustic wave generated through light irradiation, an optical energy absorption density distribution derived from an initial sound pressure distribution, an absorption coefficient distribution, a concentration distribution of a substance forming tissues, or the like. The concentration distribution of a substance is, for example, an oxygen saturation distribution, an oxidized or reduced hemoglobin concentration distribution, or the like. The information on the subject may be obtained not only in the form of numerical data but also in the form of distribution information at each site within a subject. In other words, distribution information, such as an absorption coefficient distribution and an oxygen saturation distribution, may be obtained in the form of image data.

A subject information obtaining apparatus of an exemplary embodiment may employ an ultrasonic echo technique in which the apparatus irradiates a subject with an ultrasonic wave and receives a wave reflected inside the subject so as to obtain information on the subject. In the case of such an apparatus that employs the ultrasonic echo technique, the information to be obtained on the subject is characteristic information that reflects a difference in acoustic impedance among tissues within the subject.

In each of the exemplary embodiments described hereinafter, a subject information obtaining apparatus includes a holder, a receiver, a shape detector, and a shape controlling unit.

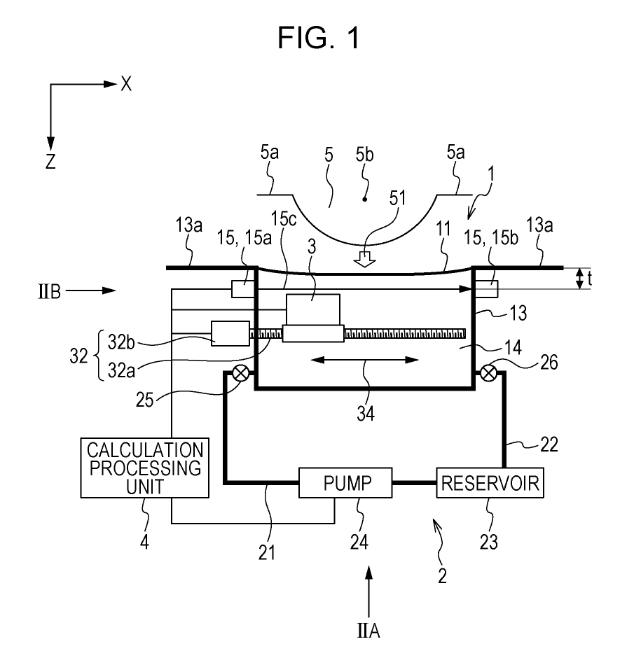

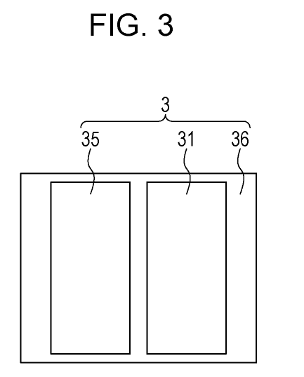

For example, referring to FIG. 1, the holder serves to hold a subject 5, and a holding member 11 corresponds to the holder, which will be described later in detail. The receiver serves to receive, through the holder, an elastic wave, or specifically an ultrasonic wave, emitted from the subject 5 held by the holder, and a probe 31 (see FIG. 3) of a probe unit 3 corresponds to the receiver. The shape detector serves to detect the shape of the holder, and a photo-interrupter 15 corresponds to the shape detector. The shape controlling unit serves to control the shape of the holder on the basis of a result of detection by the shape detector, and a matching liquid adjusting unit 2 corresponds to the shape controlling unit.

The matching liquid adjusting unit 2 serving as the shape controlling unit controls the shape of the holding member 11 serving as the holder on the basis of the result of detection by the photo-interrupter 15 serving as the shape detector, and thus the thickness of the subject 5 held by the holding member 11 can be adjusted to a predetermined thickness. Consequently, an S/N ratio of a signal that is based on a received elastic wave can be improved.

More specifically, with the apparatus discussed in Japanese Patent Laid-Open No. 2007-282960, the tensile force of the film serving as a holder is measured, and the pressure on a subject is controlled more precisely on the basis of the result of the measurement so as to be kept constant, and thus the thickness of the subject cannot be reduced to or below a predetermined value (fixed value). Since the pressure is fixed, the thickness of the subject differs depending on the size of the subject (i.e., a larger size leads to a greater thickness). In other words, even when the tensile force of the film is detected, it is difficult to determine the shape, or specifically, the thickness of the subject, and the shape, or specifically, the thickness of the subject cannot be controlled to or below a predetermined value by controlling the applied pressure to be constant regardless of the size of the subject.

On the other hand, with the exemplary embodiments, the shape of the holder (film) which directly reflects the shape (thickness) of the subject is detected, and the shape controlling unit is controlled on the basis of the result of the detection (i.e., the shape (thickness) of the subject). Thus, the thickness of the subject can be controlled to or below a predetermined thickness. More specifically, in the exemplary embodiments, instead of keeping the pressure to be applied to the subject constant regardless of the size of the subject, when the size of the subject is large, for example, the shape controlling unit is controlled so as to increase the pressure to be applied to the subject, and thus the thickness of the subject can be controlled to a predetermined thickness.

As a result, attenuation of an ultrasonic wave inside a subject can be suppressed, or in the case of an apparatus that utilizes photoacoustic effects, attenuation of light can be suppressed. Thus, the S/N ratio of a signal that is based on a received elastic wave (hereinafter, simply referred to as an elastic wave signal or an ultrasonic wave signal in some cases) can be improved. The term thickness corresponds to the size of a subject along a direction indicated by an arrow 51 in FIG. 1 (i.e., a direction along which the directivity of the probe 31 serving as the receiver is high and which corresponds to a Z direction). Hereinafter, various exemplary embodiments, or specifically, the modes for detecting and controlling the shape of a subject will be described in detail in each of the exemplary embodiments.

### First Exemplary Embodiment

FIG. 1 schematically illustrates an exemplary configuration of a subject information obtaining apparatus according to a first exemplary embodiment.

A holding unit 1 has a watertight structure formed by the holding member 11 that holds the subject 5 and a receptacle 13, and the holding member 11 and the receptacle 13 form a sealed container. The holding member 11 is preferably formed by a member having an acoustic impedance (1.5 to 1.6×10⁶ kg/m²sec) that is substantially the same as the acoustic impedance of the subject 5 or the probe 31, which will be described later, and in the case of an apparatus that utilizes photoacoustic effects, the holding member 11 is preferably formed by a member having high light transmittance (preferably, 90% or higher). Specific materials that match the above description include silicone rubber, urethane rubber, styrene-based elastomer, and olefin-based elastomer. A flexible material, such as rubber, has an advantage in that such a material is less likely to be wrinkled when holding the subject 5.

The holding member 11 is preferably thin enough to suppress attenuation of an ultrasonic wave. Preferably, the holding member 11 may be formed to have a thickness that is one-fourth or less of the wavelength of the ultrasonic wave, so that reflection of the ultrasonic wave can be prevented and noise that negatively affects image reconstruction can be reduced.

The sealed container formed by the holding member 11 and the receptacle 13 is filled with an acoustic matching liquid 14. The acoustic matching liquid 14 serves to achieve acoustic impedance matching between the holding member 11 serving as the holder and the probe 31 serving as the receiver. Here, if the air is present, information may not be obtained through the ultrasonic wave, and thus it is preferable that measures be taken to remove the air at least from an information obtaining region.

The receptacle 13 is preferably provided with a chest wall receiving surface 13a that receives a chest wall 5a of the subject 5 when the holding member 11 holds the subject 5, as illustrated in FIG. 1. The receptacle 13 has such strength that the receptacle 13 does not deform when the holding member 11 holds the subject 5 and when the shape of the holding member 11 is controlled, which will be described later.

The photo-interrupter 15 is provided on external side surfaces of the receptacle 13 for detecting the shape of the holding member 11. The photo-interrupter 15 includes a light-emitting side photo-interrupter 15a and a light-receiving side photo-interrupter 15b, which are provided opposing each other with the receptacle 13 located therebetween. Part of the receptacle 13 allows light to pass therethrough so that light from the light-emitting side photo-interrupter 15a can be received by the light-receiving side photo-interrupter 15b. The direction in which light travels in the photo-interrupter 15, or in other words, a direction 15c in which the light is detected is orthogonal to a direction along which the directivity of the probe 31 is high, or in other words, the direction 51 along which the directivity of the receiver is high.

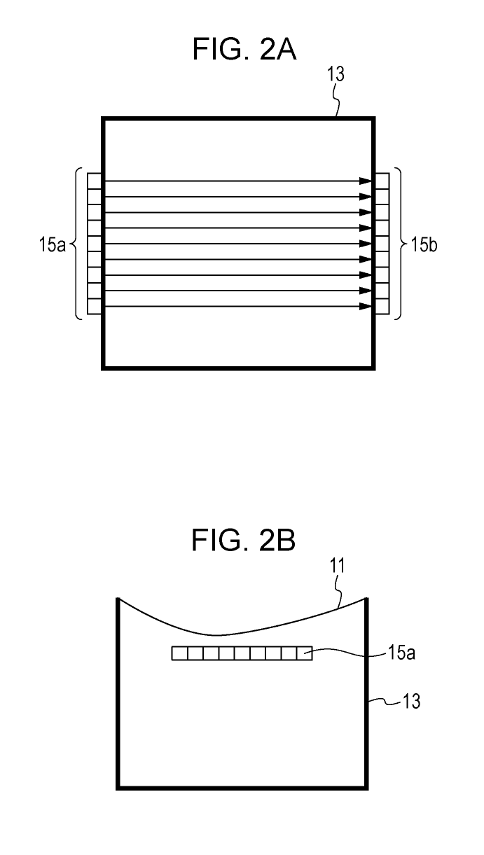

FIGS. 2A and 2B schematically illustrate a configuration in which a plurality of photo-interrupters 15 is provided. FIG. 2A illustrates the subject information obtaining apparatus of the first exemplary embodiment, as viewed in a direction IIA (i.e., Z direction) of FIG. 1. FIG. 2B illustrates the subject information obtaining apparatus of the first exemplary embodiment, as viewed in a direction IIB (i.e., X direction) of FIG. 1. A maximum deformation position of the holding member 11 does not always fall at the center thereof depending on the position at which the holding member 11 holds the subject 5.

Thus, providing the plurality of photo-interrupters 15, as illustrated in FIGS. 2A and 2B, makes it possible to detect the shape of the holding member 11 (i.e., the thickness of the subject 5) even when the maximum deformation position of the holding member 11 is offset from the center thereof. In the first exemplary embodiment, a detecting position t of the photo-interrupter 15 is located 40 mm from the chest wall receiving surface 13a. When light from the light-emitting side photo-interrupter 15a does not reach the light-receiving side photo-interrupter 15b, it can be determined that the holding member 11 has deformed by 40 mm or more.

Note that the detecting position t may be decided, as appropriate, with the frequency of the probe 31 to be used or attenuation of the acoustic wave through the acoustic matching liquid 14 taken into consideration, and thus the distance between the chest wall receiving surface 13a and the detecting position t is not limited to 40 mm. Alternatively, a camera may be used as an alternative shape detector, and the shape of the holding member 11 may be determined by processing a captured image.

The amount of the acoustic matching liquid 14 in the sealed container formed by the holding member 11 and the receptacle 13 is adjusted by the acoustic matching liquid adjusting unit 2 serving as the shape controlling unit for the holding member 11. Note that the volume of the sealed container is substantially equivalent to the volume of the receptacle 13, and thus the sealed container may simply be indicated as the receptacle 13 in the descriptions to follow.

The acoustic matching liquid adjusting unit 2 includes a supply pipe 21, a discharge pipe 22, a reservoir 23, a pump 24, a supply cock 25, and a discharge cock 26, and the supply pipe 21 and the discharge pipe 22 are connected to the receptacle 13. The reservoir 23 stores the acoustic matching liquid 14 in an amount that is sufficient to adjust the amount of the acoustic matching liquid 14 within the receptacle 13. Driving the pump 24 causes the acoustic matching liquid 14 in the reservoir 23 to be supplied to the receptacle 13 through the supply pipe 21 and the supply cock 25. In addition, the acoustic matching liquid 14 is collected into the reservoir 23 from the receptacle 13 through the discharge cock 26 and the discharge pipe 22. The supply cock 25 and the discharge cock 26 serve to release or block the flow of the acoustic matching liquid 14. Driving the pump 24 while the supply cock 25 is opened and the discharge cock 26 is closed can increase the amount of the acoustic matching liquid 14 within the receptacle 13. When the amount of the acoustic matching liquid 14 within the receptacle 13 is to be reduced, the pump 24 is stopped, and the discharge cock 26 is opened. Since the sealed container (watertight structure) formed by the receptacle 13 and the holding member 11 is filled with the acoustic matching liquid 14, the shape of the holding member 11 can be controlled by adjusting the amount of the acoustic matching liquid 14 within the receptacle 13. Alternatively, the amount of the acoustic matching liquid 14 within the receptacle 13 may be controlled by adjusting the openings of the supply cock 25 and the discharge cock 26 while the pump 24 is driven constantly.

The probe unit 3 is provided inside the receptacle 13, and the probe unit 3 receives an ultrasonic wave generated as the subject 5 is irradiated with light from a light source (not illustrated). FIG. 3 illustrates the configuration of the probe unit 3, as viewed from a side at which the holding member 11 is provided. The probe unit 3 includes the probe 31 that receives an ultrasonic wave and a light irradiation unit 35 that is irradiated with light from the light source (not illustrated), and the probe 31 and the light irradiation unit 35 are integrated by a housing 36. The probe unit 3 is moved along a direction of an arrow 34 by a scanning mechanism 32, which serves as a receiving position controlling unit that controls the position of the probe 31 serving as the receiver relative to the holding member 11 serving as the holder. The scanning mechanism 32 includes an actuator 32a in which a leading screw mechanism and a guide are integrated and a motor 32b serving as a power source for driving the scanning mechanism 32. The scanning direction is not limited to a single direction, and a two-dimensional scan or a curved scan may be carried out. Light emitted from the light irradiation unit 35 passes through the acoustic matching liquid 14 and the holding member 11 and reaches the subject 5. The light is then scattered and absorbed inside the subject 5 and reaches an information obtaining site 5b. An ultrasonic wave is then generated at the information obtaining site 5b, and the generated ultrasonic wave passes through the subject 5, the holding member 11, and the acoustic matching liquid 14 and is received by the probe 31.

A calculation processing unit 4 is formed typically by a workstation or the like and carries out noise reduction processing and so forth on an electric signal obtained from the probe 31 so as to reconstruct an image. In addition, the calculation processing unit 4 detects information on the probe unit 3 and the photo-interrupter 15 and carries out overall processing, such as controlling the acoustic matching liquid adjusting unit 2, accordingly.

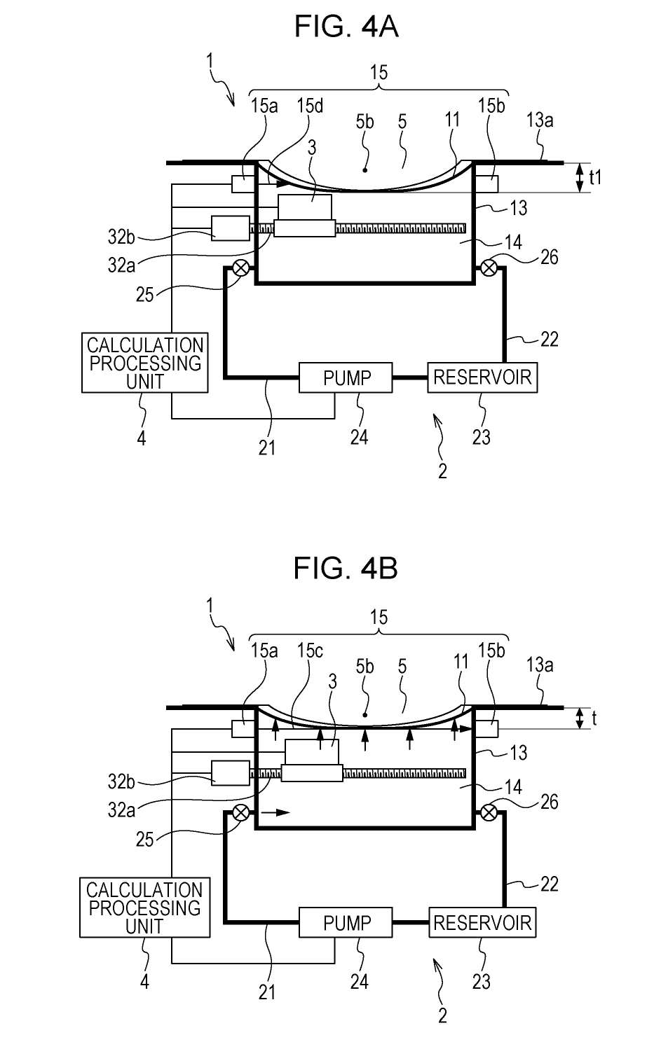

FIGS. 4A and 4B illustrate the states of the holding member 11 holding the subject 5 before and after the shape of the holding member 11 is detected and controlled. FIG. 4A illustrates the shape of the holding member 11 held before the shape thereof is controlled, and FIG. 4B illustrates the shape of the holding member 11 obtained after the shape thereof has been controlled.

When the holding member 11 holds the subject 5, deflection t1 is generated in the holding member 11 due to the retaining force of the subject 5. While the amount of such deflection t1 is, for example, equal to or greater than 40 mm, the light from the light-emitting side photo-interrupter 15a is blocked by the holding member 11 as indicated by an arrow 15d. In other words, the stated case indicates that the total thickness of the holding member 11 and the subject 5 is equal to or greater than 40 mm, and in a case in which the information obtaining site 5b is located in the vicinity of the chest wall 5a (see FIG. 1) in the aforementioned stated, a sufficient S/N ratio of an ultrasonic wave signal necessary to obtain the information cannot be achieved. When the shape of the holding member 11 is detected as the light from the light-emitting side photo-interrupter 15a is blocked, the acoustic matching liquid adjusting unit 2 controls the shape of the holding member 11, or in other words, the shape, or specifically, the thickness of the subject 5. In the aforementioned case, the holding member 11 needs to be lifted against the subject 5. The holding member 11 can be lifted by increasing the amount of the acoustic matching liquid 14 within the receptacle 13. Thus, as described above, the pump 24 is driven, the supply cock 25 is opened, and the discharge cock 26 is closed. Since the receptacle 13 is strong enough not to deform through the shape control, the acoustic matching liquid 14 that has flowed into the receptacle 13 lifts the holding member 11 so as to pressurize the subject 5. Once the holding member 11 is lifted to a level at which the holding member 11 is not detected by the photo-interrupter 15, information can start being obtained. Processing covering from the shape detection to the shape control may be carried out automatically with the calculation processing unit 4. Alternatively, the apparatus may be configured to allow an operator using the apparatus to recognize the state of the shape detection and may control the shape of the holding member 11 in accordance with an instruction from the operator inputted through an operation button provided for the shape control. As another alternative, the shape may be detected continuously, and feedback control may be carried out with the calculation processing unit 4.

Through the control described above, even if the information obtaining site 5b is located deep inside the subject 5 while the subject 5 is not held by the holding member 11, holding the subject 5 in a pressurized manner by the holding member 11 formed by a thin member makes it possible to reduce the total thickness of the holding member 11 and the subject 5. In other words, attenuation of the ultrasonic wave can be suppressed, and thus the S/N ratio of the ultrasonic wave signal can be improved.

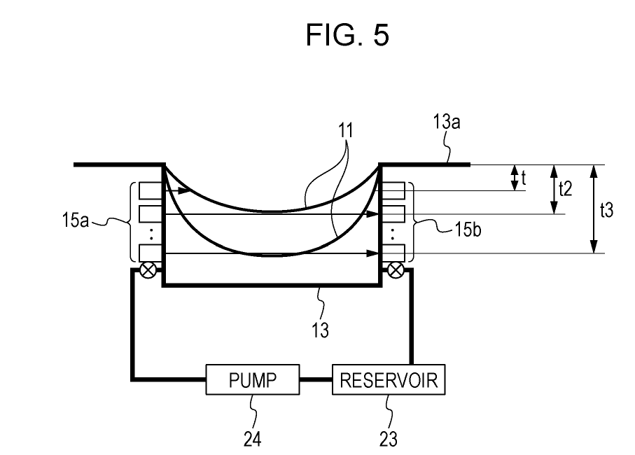

FIG. 5 illustrates the configuration of the shape detector in which the plurality of photo-interrupters 15 are arranged in a direction parallel to the direction along which the directivity of the probe 31 is high. In this configuration, the photo-interrupters 15 are provided at positions that are more spaced apart from the chest wall receiving surface 13a than a maximum position t at which a sufficient S/N ratio of an ultrasonic wave signal necessary for obtaining information can be achieved. This configuration makes it possible to detect how much the holding member 11 has deflected when the deflection of the holding member 11 holding the subject 5 has exceeded the maximum position t and the shape control has become necessary. When an amount by which the shape of the holding member 11 needs to be controlled is large, or in other words, when the deflection amount of the holding member 11 is extremely large, the subject 5 is pressurized that much more, and thus a load on the examinee increases. Thus, although the shape needs to be controlled to the maximum position t in order to obtain a sufficient S/N ratio, a maximum amount by which the shape control is permitted may be set with a load on an examinee taken into consideration. For example, if the deflection amount of the holding member 11 corresponds to t3 prior to the shape control, the permitted shape control amount may be set up to t2. In addition, aside from setting the amount by which the shape is controlled, by adjusting the pump 24 and the supply cock 25, the speed at which the holding member 11 is deformed may be changed in accordance with the position at which the shape is detected. For example, the shape may be controlled at a high speed from an initial detecting position to a subsequent detecting position, and the speed at which the shape is controlled may be reduced so as to move to the following detecting position more slowly. In other words, the speed at which the shape of the holding member 11 is changed may be changed stepwise. Note that the positions at which the plurality of photo-interrupters 15 are provided may be closer to the chest wall receiving surface 13a than the maximum position t.

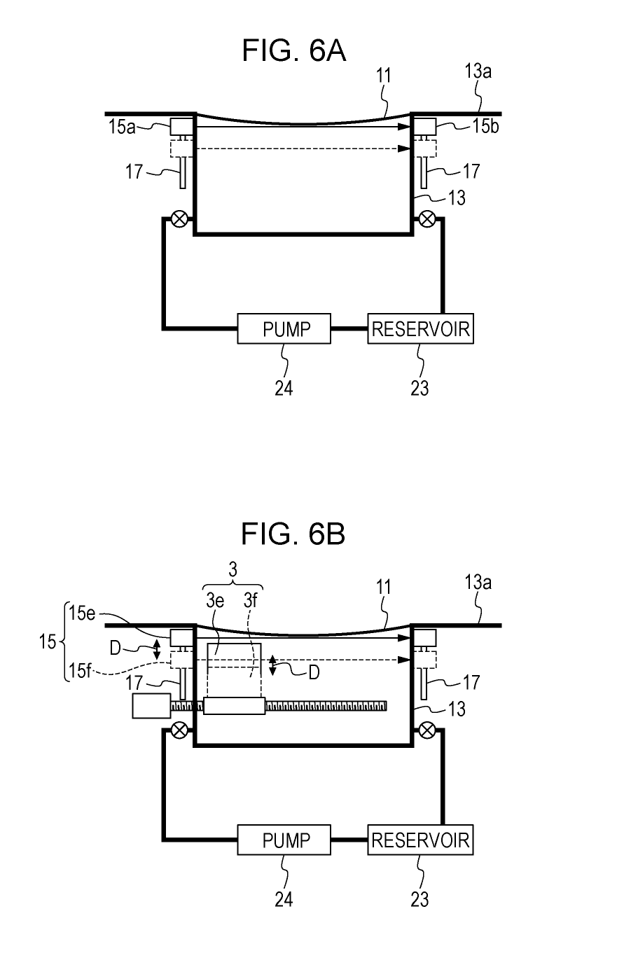

FIG. 6A schematically illustrates a configuration that includes a detecting position controlling unit that controls the detecting position of the shape detector relative to the holder. The light-emitting side photo-interrupter 15a and the light-receiving side photo-interrupter 15b are provided on linear guides 17 (corresponding to the detecting position controlling units) that can move the light-emitting side photo-interrupter 15a and the light-receiving side photo-interrupter 15b along a direction in which the directivity of the probe 31 is high. Thus, the photo-interrupter 15 can be moved by a driving mechanism (not illustrated) along a direction in which the directivity of the probe 31 is high. In addition, an encoder (not illustrated) is provided to measure the distance between the photo-interrupter 15 and the chest wall receiving surface 13a. The S/N ratio of the ultrasonic wave signal improves as the subject 5 is thinner. It may be possible to further reduce the thickness of the subject 5 to less than 40 mm depending on the size or the firmness of the subject 5. With the above configuration, the position of the photo-interrupter 15 is controlled by the calculation processing unit 4 in accordance with the distance between the photo-interrupter 15 and the chest wall receiving surface 13a detected with the encoder. The shape of the holding member 11 is controlled with the acoustic matching liquid adjusting unit 2 in accordance with the position of the photo-interrupter 15. In other words, the shape of the holding member 11 can be controlled so as to follow the position of the photo-interrupter 15. The position of the photo-interrupter 15 can be controlled by an operator using the apparatus operating an operation button (not illustrated). In addition, the distance between the photo-interrupter 15 and the chest wall receiving surface 13a is displayed so as to allow the operator to recognize the distance. According to such a configuration, it is possible not only to determine whether or not the total thickness of the holding member 11 and the subject 5 is equal to or greater than 40 mm but also to control the shape of the holding member 11 in such a manner that allows the subject 5 to have a desired thickness.

FIG. 6B schematically illustrates a configuration that includes a receiving position controlling unit that controls the position of the receiver relative to the holder. Through this configuration, the probe unit 3 can be moved along a direction in which the directivity of the probe 31 is high in accordance with the movement of the photo-interrupter 15. Light emitted from the light irradiation unit 35 of the probe unit 3 is attenuated by the acoustic matching liquid 14. Therefore, it is preferable that the distance between the light irradiation unit 35 and the holding member 11 be small. With this configuration, the probe unit 3 is provided at a position at which the probe unit 3 does not block the light in the photo-interrupter 15. When the photo-interrupter 15 is located at a position 15e, the probe unit 3 is located at a position 3e. The probe unit 3 is moved by an amount that is equivalent to the amount by which the photo-interrupter 15 is moved. When the photo-interrupter 15 is moved by an amount D and is located at a position 15f, the probe unit 3 is also moved by the amount D and is located at a position 3f accordingly. Thus, the positional relationship among the light in the photo-interrupter 15, the probe unit 3, and the maximum displacement position of the holding member 11 stays constant, and the attenuation of the light due to the deformation of the holding member 11 and the shape control (i.e., attenuation due to the distance between the holding member 11 and the probe unit 3) can be suppressed. The configuration in which the probe unit 3 is moved can also be applied to the above-described configuration in which the plurality of photo-interrupters 15 are provided.

Through the configuration described above, the shape of the holding member 11 can be controlled in a more detailed manner.

In addition, configuring the apparatus such that the position of the shape detector can be changed makes it possible to control the total thickness of the holding member 11 and the subject 5 to a desired thickness, and the S/N ratio of the ultrasonic wave signal necessary for obtaining information can be improved.

### Second Exemplary Embodiment

In a second exemplary embodiment, part of the receptacle is formed by a deformable member, and the shape of the holding member 11 is controlled by changing the shape of the deformable member. Configurations that are identical to those of the first exemplary embodiment will be given identical reference characters, and descriptions thereof will be omitted.

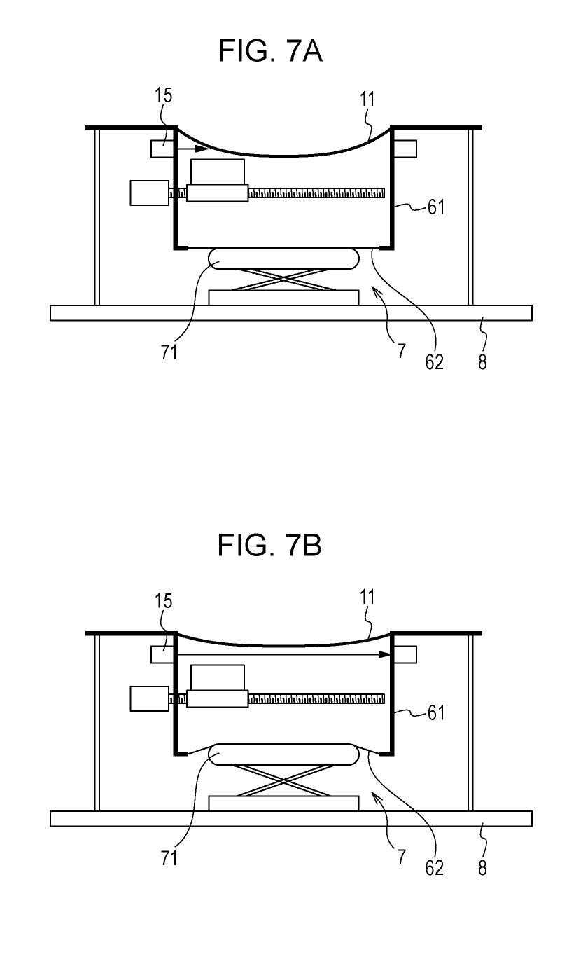

FIGS. 7A and 7B schematically illustrate an exemplary configuration of a subject information obtaining apparatus according to the second exemplary embodiment. FIG. 7A illustrates the holding member 11 before the shape thereof is controlled, and FIG. 7B illustrates the holding member 11 after the shape thereof has been controlled. A receptacle is formed by a frame 61 and a deformable member 62 serving as a base. The frame 61 and the deformable member 62 form a watertight structure that can sealingly hold the acoustic matching liquid 14 thereinside. In addition, as in the receptacle 13 of the first exemplary embodiment, the frame 61 has such strength that the frame 61 does not deform when the holding member 11 holds the subject 5 and the shape of the holding member 11 is controlled, which will be described later. Although the deformable member 62 serves as the base of the receptacle in the second exemplary embodiment, the deformable member 62 may be provided as a side wall of the receptacle, and the deformable member 62 is not particularly limited to serve as the base. An elevator 7 is provided underneath the deformable member 62 so as to push up and deform the deformable member 62, and the elevator 7 corresponds to the shape controlling unit in the second exemplary embodiment. The elevator 7 is a pantograph type elevator and includes an abutting member 71 that abuts against the deformable member 62. The elevator 7 moves the abutting member 71 up and down with an electric motor (not illustrated). The frame 61 and the elevator 7 are coupled to a base plate 8. Thus, by moving the abutting member 71 upward by driving the elevator 7 so as to push up the deformable member 62, the amount by which the deformable member 62 deforms can be controlled. The shape of the holding member 11 is detected with the photo-interrupter 15, as in the first exemplary embodiment. The elevator 7 is controlled by the calculation processing unit 4 (not illustrated in FIGS. 7A and 7B) in accordance with the detection of the photo-interrupter 15.

When the holding member 11 deforms by holding the subject 5 and the photo-interrupter 15 detects the holding member 11, the holding member 11 needs to be controlled. Specifically, the deformable member 62 is pushed up by controlling the elevator 7. Since the receptacle formed by the frame 61 and the deformable member 62 is filled with the acoustic matching liquid 14, pushing the deformable member 62 upward causes the pressure of the acoustic matching liquid 14 within the receptacle to rise. Since the frame 61 is strong enough not to deform, the holding member 11 is pushed upward so as to pressurize the subject 5. In this manner, the shape of the holding member 11 can be controlled by changing the pressure of the acoustic matching liquid 14. The holding member 11 is thus pushed upward until the holding member 11 has a shape that is not detected by the photo-interrupter 15.

Through the control described above, even if an information obtaining site is located deep inside the subject 5 while the subject 5 is not held by the holding member 11, holding the subject 5 by the holding member 11 formed by a thin member while controlling the shape of the holding member 11 makes it possible to reduce the total thickness of the holding member 11 and the subject 5. In other words, attenuation of the ultrasonic wave can be suppressed, and the S/N ratio of the ultrasonic wave signal can thus be improved.

In the second exemplary embodiment as well, the shape of the holding member 11 may be detected not only by determining whether or not the aforementioned total thickness is equal to or greater than 40 mm but also by using the alternative detection methods described in the first exemplary embodiment.

### Third Exemplary Embodiment

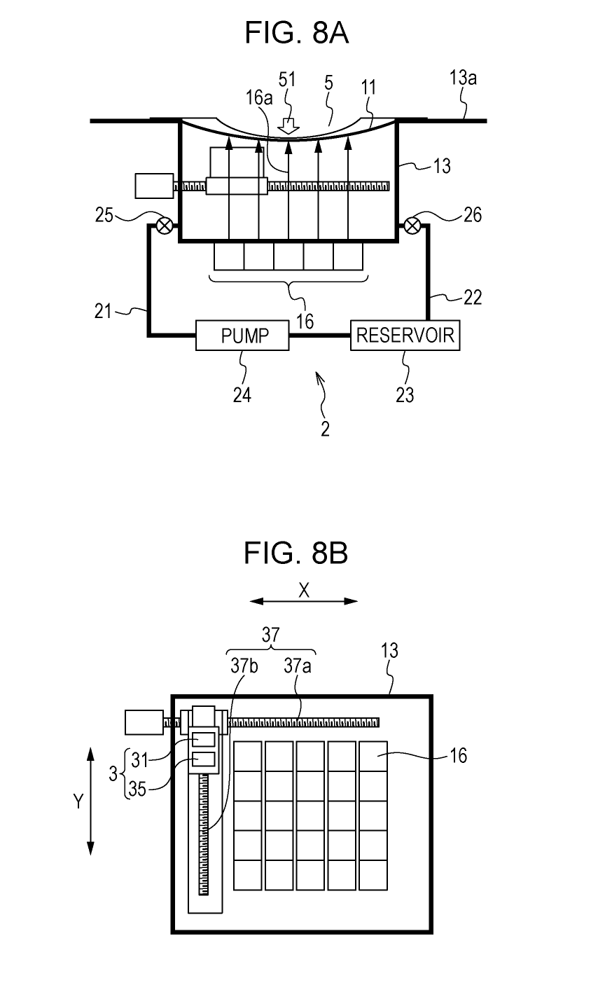

In a third exemplary embodiment, the shape of the holder is detected in a direction parallel to the direction along which the directivity of the receiver is high, and thus a distance measuring sensor is provided to serve as the shape detector for the holding member 11. FIG. 8A schematically illustrates a configuration of a subject information obtaining apparatus according to the third exemplary embodiment. FIG. 8B schematically illustrates the configuration of the subject information obtaining apparatus according to the third exemplary embodiment, as viewed from a side at which the holding member 11 is provided. A distance measuring sensor 16 is provided on the base of the receptacle 13 so as to measure the distance between the distance measuring sensor 16 and the holding member 11. An ultrasonic sensor, for example, can be used as the distance measuring sensor 16. The probe unit 3 is moved by a scanning mechanism 37 that is capable of moving the probe unit 3 along two axes with an X direction scanning mechanism 37a and a Y direction scanning mechanism 37b. The scanning mechanism 37 serving as the receiving position controlling unit and the probe unit 3 are initially arranged at positions at which the scanning mechanism 37 and the probe unit 3 do not interfere with the distance measuring sensor 16. A direction 16a along which the distance is measured with the distance measuring sensor 16 is parallel to the direction 51 along which the directivity of the probe 31 is high. The calculation processing unit 4 (not illustrated in FIGS. 8A and 8B) calculates the distance from a plane containing the chest wall receiving surface 13a to the holding member 11 on the basis of the value obtained by the distance measuring sensor 16. Thus, the total thickness of the holding member 11 and the subject 5 while the holding member 11 holds the subject 5 can be measured. As in the first exemplary embodiment, the acoustic matching liquid adjusting unit 2 is provided to serve as the shape controlling unit.

In the third exemplary embodiment, the total thickness of the holding member 11 and the subject 5 for obtaining an S/N ratio of an ultrasonic wave signal necessary for obtaining information on the subject 5 is set to be equal to or less than 40 mm. The total thickness of the holding member 11 and the subject 5 is calculated by the calculation processing unit 4 after the holding member 11 has held the subject 5. When the calculated value is not equal to or less than 40 mm, the calculation processing unit 4 controls the acoustic matching liquid adjusting unit 2 to operate as described above so as to control the shape of the holding member 11. Since the distance measuring sensor 16 is used in the third exemplary embodiment, it is possible not only to determine whether or not the total thickness of the holding member 11 and the subject 5 is equal to or less than 40 mm but also to control the shape of the holding member 11 so as to bring the total thickness to a desired value. In addition, since the shape is detected in a direction in which the holding member 11 holds the subject 5, it is possible to obtain the surface shape of the holding member 11 along an area above the distance measuring sensor 16, and such information can be used to correct a light quantity, a distribution of the light quantity, and an acoustic wave signal when carrying out a calculation for turning subject information into an image. In addition, the techniques for controlling the shape of the holding member 11 described in the first and second exemplary embodiments can also be employed in the third exemplary embodiment. If the configuration in which the probe unit 3 is moved along a direction in which the directivity of the probe 31 is high is employed, the probe unit 3 may be moved three-dimensionally so as to follow the surface shape of the holding member 11.

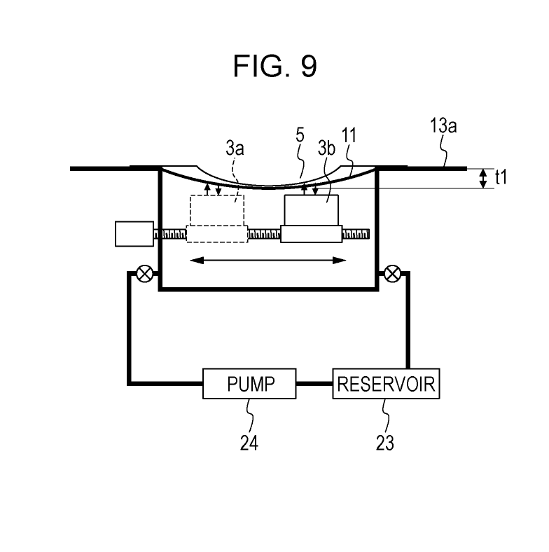

Instead of providing a separate distance measuring sensor as described above, the shape of the holding member 11 can be detected by using the receiver for obtaining information on the subject 5. FIG. 9 illustrates a configuration in which the distance between the probe 31 and the holding member 11 is measured by the probe 31. After the holding member 11 holds the subject 5, the probe unit 3 is once moved from an initial position 3a to a position 3b corresponding to an edge of the imaging area. At this time, the distance between the probe 31 and the holding member 11 is measured, and the calculation processing unit 4 calculates the distance t1 between a plane containing the chest wall receiving surface 13a and the holding member 11. If the distance t1 is not equal to or less than 40 mm, the acoustic matching liquid adjusting unit 2 is operated as described above so as to control the shape of the holding member 11. Note that the range in which the probe unit 3 is moved so as to measure the distance does not need to cover the entire surface of the holding member 11 and may cover only an area around the center at which the deflection amount is large. Through this configuration, the shape of the holding member 11 can be detected without a separate distance measuring sensor, and the configuration can thus be simplified. In addition, since the shape of the holding member 11 can be detected directly with the probe 31, the shape can be detected with high accuracy and high reliability. In the third exemplary embodiment as well, the alternative methods for controlling the shape of the holding member 11 described in the first and second exemplary embodiments can be employed.

Through the control described above, even if the information obtaining site 5b is located deep inside the subject 5 while the subject 5 is not held by the holding member 11, holding the subject 5 in a pressurized manner by the holding member 11 formed by a thin member makes it possible to reduce the total thickness of the holding member 11 and the subject 5. In other words, attenuation of the ultrasonic wave can be suppressed, and thus the S/N ratio of the ultrasonic wave signal can be improved. In addition, configuring the apparatus such that the shape of the holding member 11 is detected by measuring the distance in a direction parallel to the direction along which the directivity of the probe is high allows the total thickness of the holding member 11 and the subject 5 to be controlled to a desired thickness, and the S/N ratio of the ultrasonic wave signal necessary for obtaining information can be improved.

According to the above-described exemplary embodiments, an S/N ratio of a signal that is based on a received elastic wave can be improved.

While the present disclosure has been described with reference to exemplary embodiments, it is to be understood that these exemplary embodiments are not seen to be limiting. The scope of the following claims is to be accorded the broadest interpretation so as to encompass all such modifications and equivalent structures and functions.

This application claims the benefit of Japanese Patent Application No. 2013-125065 filed Jun. 13, 2013, which is hereby incorporated by reference herein in its entirety.

What is claimed is:

1. A subject information obtaining apparatus, comprising:
a holder configured to hold a subject;
a receiver configured to receive, through the holder, an elastic wave emitted from the subject held by the holder;
at least one shape detector configured to detect a shape of the holder; and
a shape controlling unit configured to control the shape of the holder based on a result of detection by the at least one shape detector.

2. The subject information obtaining apparatus according to claim 1,
wherein the shape controlling unit controls the shape of the holder so as to change a thickness of the subject.

3. The subject information obtaining apparatus according to claim 1,
wherein the at least one shape detector detects the shape of the holder in a direction orthogonal to a direction along which directivity of the receiver is high.

4. The subject information obtaining apparatus according to claim 3,
wherein the at least one shape detector includes a plurality of shape detectors, and
wherein the plurality of shape detectors are arranged in a direction parallel to the direction along which the directivity is high.

5. The subject information obtaining apparatus according to claim 3,
wherein the at least one shape detector includes a plurality of shape detectors, and
wherein the plurality of shape detectors are arranged in a direction orthogonal to the direction along which the directivity is high.

6. The subject information obtaining apparatus according to claim 1,
wherein the at least one shape detector detects the shape of the holder in a direction parallel to a direction along which directivity of the receiver is high.

7. The subject information obtaining apparatus according to claim 1, further comprising:
a receiving position controlling unit configured to control a position of the receiver relative to the holder.

8. The subject information obtaining apparatus according to claim 1, further comprising:
a detecting position controlling unit configured to control a position of the at least one shape detector relative to the holder.

9. The subject information obtaining apparatus according to claim 1, further comprising:
a receptacle, the receptacle and the holder forming a sealed container,
wherein the sealed container is filled with a matching liquid for achieving acoustic impedance matching between the holder and the receiver, and
wherein the shape controlling unit controls the shape of the holder by adjusting an amount of the matching liquid.

10. The subject information obtaining apparatus according to claim 9,
wherein a portion of the receptacle is formed by a deformable member, and
wherein the shape controlling unit controls the shape of the holder by controlling a deformation amount of the deformable member so as to change a pressure of the matching liquid inside the sealed container.

11. The subject information obtaining apparatus according to claim 9,
wherein the shape controlling unit controls the shape of the holder while changing a speed at which the shape controlling unit changes the shape of the holder stepwise.

12. The subject information obtaining apparatus according to claim 9,
wherein the receiver is disposed inside the sealed container.

13. The subject information obtaining apparatus according to claim 9,
wherein the receiving position controlling unit controls the position of the receiver based on a result of detection by the at least one shape detector.
